(19) United States
(12) Patent Application Publication
(10) Pub. No.: US 2022/0015632 A1
(43) Pub. Date: Jan. 20, 2022

(54) A SYSTEM AND A METHOD FOR MEASURING PRESSURE OF AN EYE

(71) Applicant: PHOTONO OY, Helsinki (KR)

(72) Inventors: Antti KONTIOLA, Helsinki (FI); Ari SALMI, Helsinki (FI); Edward HÆGGSTRÖM, Helsinki (FI); Daniel Veira CANLE, Helsinki (FI); Henri MALINEN, Helsinki (FI); Joni MÄKINEN, Helsinki (FI)

(21) Appl. No.: 17/297,851

(22) PCT Filed: Nov. 20, 2019
(86) PCT No.: PCT/FI2019/050828

§ 371 Date: May 27, 2021

(30) Foreign Application Priority Data
Nov. 29, 2018 (FI) 20186011

(51) Int. Cl. A61B 3/16; A61B 3/10

(52) U.S. Cl. CPC A61B 3/165; A61B 2562/0247; A61B 2560/02; A61B 3/102

(57) ABSTRACT

A system for measuring pressure of an eye includes an excitation source for producing a travelling air vortex ring and for directing the travelling air vortex ring to the eye, a detector for detecting an interaction between the travelling air vortex ring and a surface of the eye, and a processing device for determining an estimate of the pressure of the eye based on the detected interaction between the travelling air vortex ring and the surface of the eye. The travelling air vortex ring is produced by directing an air pressure pulse into a flow guide, and the air pressure pulse is generated with an electric spark or otherwise so that no swinging mass, such as a piston, is needed. This is advantageous especially in a case of a handheld device because a swinging mass would tend to adversely move the handheld device during a measurement.

### BACKGROUND OF THE INVENTION

### Field of the Invention

The disclosure relates to a system for measuring pressure of an eye of a human or an animal. Furthermore, the disclosure relates to a method for measuring pressure of an eye.

### Description of the Related Art

Intraocular pressure “IOP” plays a major role in the pathogenesis of the open angle glaucoma, one of the leading causes of blindness. There globally are millions of people with the open angle glaucoma, about half of which are unknowingly affected and without diagnosis. The prevalence of open angle glaucoma increases with the aging of the human population and it is expected that this will increase by 30% the number of open angle glaucoma cases during the next decade. The way to currently treat open angle glaucoma is by lowering the intraocular pressure. An eye pressure measurement is a practical way of screening the open angle glaucoma. However, screening large parts of the population is needed to find undiagnosed cases. The other type of glaucoma is narrow angle glaucoma that causes a sudden eye pressure increase that may cause blindness in a few days. Since one per mille of the population is affected with the acute narrow angle glaucoma, it would be advantageous to screen acute narrow angle glaucoma by measuring the eye pressure at health centers and other sites of the general health care as well as in the private health care sector. Therefore, it would be beneficial if every practitioner office had a system for measuring the eye pressure quickly and easily.

Contact methods such as e.g. Goldmann tonometry and Mackay-Marg tonometry for measuring eye pressure mostly require a local anesthetic to carry out the measurement and are thus impractical e.g. for screening large human populations. Non-contacting air impulse tonometers have been on the market for decades. A drawback of these tonometers is discomfort experienced by a human or animal whose eye pressure is being measured due to an air impulse directed towards and striking the eye. The publication U.S. Pat. No. 6,030,343 describes a method that is based on an airborne ultrasonic beam that is reflected from a cornea. Excitation is done by a narrow band ultrasonic tone burst that deforms the cornea, and the phase shift of an ultrasonic tone burst reflected off the deformed cornea is measured to obtain an estimate of the eye pressure. Publications US2004193033 and U.S. Pat. No. 5,251,627 describe non-contact measurement methods based on acoustic and ultrasonic excitations. It is also possible to use a shock wave, i.e. a disturbance moving faster than the speed of sound, for excitation and to estimate eye pressure based on a response caused by the shock wave on a surface of an eye.

An inconvenience related to many of the above-described non-contact eye pressure measurement methods is that in practice an excitation device such as e.g. a shock wave source needs to be quite near to an eye to achieve suitable excitation on the surface of the eye, and this may in some cases lead to discomfort experienced by a human or animal whose eye pressure is being measured.

### SUMMARY OF THE INVENTION

The following presents a simplified summary to provide basic understanding of some aspects of different invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying and non-limiting embodiments of the invention.

In this document, the word “geometric” when used as a prefix means a geometric concept that is not necessarily a part of any physical object. The geometric concept can be for example a geometric point, a straight or curved geometric line, a geometric plane, a non-planar geometric surface, a geometric space, or any other geometric entity that is zero, one, two, or three dimensional.

In accordance with the invention, there is provided a new system for measuring the pressure of an eye. The measured pressure is typically the intraocular pressure “IOP” of the eye. A system according to the invention comprises:
an excitation source for producing a travelling air vortex ring and for directing the travelling air vortex ring to the eye,
a detector for detecting an interaction between the travelling air vortex ring and a surface of the eye, and
a processing device for determining an estimate of the pressure of the eye based on the detected interaction between the travelling air vortex ring and the surface of the eye.

The excitation source comprises an air pressure pulse source and a flow guide for forming the travelling air vortex ring. The air pressure pulse source comprises one of the following: i) a chamber connected to the flow guide and containing a spark gap for producing an air pressure pulse by an electric spark, ii) a chamber connected to the flow guide and containing chemical substances for producing an air pressure pulse by a chemical reaction between the chemical substances, iii) a laser source for producing a plasma expansion in a chamber connected to the flow guide to produce an air pressure pulse.

In a system according to the invention, the air pressure pulse, and thereby the travelling air vortex ring, is generated without a swinging element that has a significant mass, e.g. a piston or an element for moving a membrane. Thus, a measurement carried out with the system according to the invention is not disturbed by a swinging mass. This is advantageous especially in a case of a handheld device because a swinging mass would tend to adversely move the handheld device during a measurement.

The travelling air vortex ring can be for example a poloidal air vortex ring that is a region where air spins around a geometric axis line that forms a closed loop. A poloidal air vortex ring tends to move in a direction that is perpendicular to the plane of the air vortex ring and so that air on the inner edge of the air vortex ring moves faster forward than air on the outer edge. The speed difference is caused by the spinning of the air around the above-mentioned geometric axis line forming the closed loop. The air vortex ring can travel up to 30 cm, or longer, in air whereas the travelling distance of e.g. a shock wave is up to 20 mm. Thus, the excitation source of the above-described device according to the invention can be significantly farther from an eye than e.g. an excitation source that produces a shock wave.

In accordance with the invention, there is provided also a new method for measuring the pressure of an eye. A method according to the invention comprises:
producing a travelling air vortex ring and directing the travelling air vortex ring to the eye,
detecting an interaction between the travelling air vortex ring and a surface of the eye, and
determining an estimate of the pressure of the eye based on the detected interaction between the travelling air vortex ring and the surface of the eye.

The travelling air vortex ring is produced by directing an air pressure pulse into a flow guide. The air pressure pulse is generated with one of the following: i) an electric spark in a chamber connected to the flow guide, ii) a chemical reaction between chemical substances in a chamber connected to the flow guide, iii) a laser source producing a plasma expansion in a chamber connected to the flow guide.

Various exemplifying and non-limiting embodiments are described in accompanied dependent claims.

Exemplifying and non-limiting embodiments both as to constructions and to methods of operation, together with additional objects and advantages thereof, are best understood from the following description of specific exemplifying embodiments when read in conjunction with the accompanying drawings.

The verbs “to comprise” and “to include” are used in this document as open limitations that neither exclude nor require the existence of un-recited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF THE DRAWINGS

Exemplifying and non-limiting embodiments of the invention and their advantages are explained in greater detail below with reference to the accompanying drawings, in which:

FIG. 1 illustrates a system according to an exemplifying and non-limiting embodiment for measuring pressure of an eye,

FIGS. 2a-2c illustrate details of systems according to exemplifying and non-limiting embodiments for measuring pressure of an eye,

FIG. 2d illustrate a detail of an exemplifying system not belonging to the scope of the invention,

FIG. 3 illustrates a detail of an exemplifying system not belonging to the scope of the invention, and

FIG. 4 shows a flowchart of a method according to an exemplifying and non-limiting embodiment for measuring pressure of an eye.

### DESCRIPTION OF THE PREFERRED EMBODIMENTS

The specific examples provided in the description below should not be construed as limiting the scope and/or the applicability of the accompanied claims. Lists and groups of examples provided in the description below are not exhaustive unless otherwise explicitly stated.

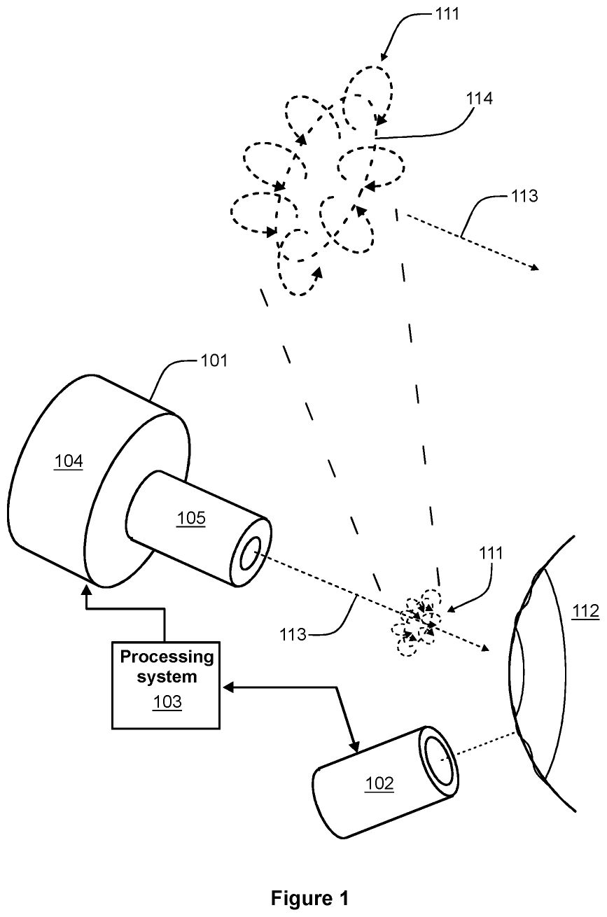

FIG. 1 illustrates a system according to an exemplifying and non-limiting embodiment for measuring pressure of an eye 112. The system comprises an excitation source 101 for producing a travelling air vortex ring 111 and for directing the travelling air vortex ring to the eye 112. The travelling air vortex ring 111 can be for example a poloidal air vortex ring that is a region where air spins around a geometric axis line 114 that forms a closed loop. A poloidal air vortex ring moves in a direction of a geometric line 113 that is perpendicular to the plane of the air vortex ring and so that air on the inner edge of the air vortex ring moves faster forward than air on the outer edge. The speed difference is caused by the spinning of the air around the geometric axis line 114. The excitation source 101 comprises an air pressure pulse source 104 and a flow guide 105 for forming the travelling air vortex ring 111. The system comprises a detector 102 for detecting an interaction between the travelling air vortex ring 111 and a surface of the eye 112. The system comprises a processing device 103 for determining an estimate of the pressure of the eye 112 based on the detected interaction between the travelling air vortex ring 111 and the surface of the eye 112.

When the traveling air vortex ring contacts the eye, it remains in contact with the surface of the eye, e.g. a cornea, until the air vortex ring disappears. During the time the air vortex ring contacts the eye it interacts with eye causing the surface of the eye to bend and to vibrate. The bending of surface of the eye and vibration frequency can be used to deduce the pressure of the eye, e.g. the interocular pressure “IOP”. At high pressure of the eye the vibration frequency is higher than at lower pressure of the eye.

In a system according to an exemplifying and non-limiting embodiment, the detector 102 comprises means for detecting a surface wave caused by the travelling air vortex ring 111 on the surface of the eye 112. The surface wave can be e.g. a manifestation of a membrane wave caused by the travelling air vortex ring 111 on the cornea of the eye. The means for detecting a surface wave can be for example an optical interferometer, an optical coherence tomography device, a laser Doppler vibrometer, or an ultrasonic transducer. The travelling speed of the surface wave on the surface of the eye 112 depends on the pressure of the eye 112. Therefore, in this exemplifying case, the processing device 103 can be configured to estimate the pressure of the eye based on the travelling speed of the detected surface wave.

In a system according to an exemplifying and non-limiting embodiment, the detector 102 comprises means for detecting a displacement of the surface of the eye caused by the travelling air vortex ring 111. The means for detecting the displacement can be for example an optical interferometer, an optical coherence tomography device, a laser Doppler vibrometer, or an ultrasonic transducer. The oscillation rate of the displacement in the direction perpendicular to the surface of the eye 112 depends on the pressure of the eye 112. Therefore, in this exemplifying case, the processing device 103 can be configured to estimate the pressure of the eye 112 based on the oscillation rate of the detected displacement. For another example, a speed at which the surface of the eye retracts when being hit by the travelling air vortex ring depends on the pressure of the eye. Therefore, the processing device 103 can be configured to estimate the pressure of the eye based on the retraction speed of the surface of the eye. For a third example, a speed at which the retracted surface of the eye returns towards its normal position depends on the pressure of the eye. Therefore, the processing device 103 can be configured to estimate the pressure of the eye based on the speed at which the retracted surface of the eye returns towards its normal position. For a fourth example, a delay after which the retracted surface of the eye returns towards its normal position depends on the pressure of the eye. Therefore, the processing device 103 can be configured to estimate the pressure of the eye based on the delay after which the retracted surface of the eye returns towards its normal position. For a fifth example, a retraction depth of the surface of the eye when being hit by the travelling air vortex ring depends on the pressure of the eye. Therefore, the processing device 103 can be configured to estimate the pressure of the eye based on the retraction depth.

In a system according to an exemplifying and non-limiting embodiment, the detector 102 comprises a pressure sensor for detecting an air pressure transient reflected off the surface of the eye 112 when the travelling air vortex ring hits the surface of the eye. The air pressure transient depends on the pressure of the eye 112. Therefore, in this exemplifying case, the processing device 103 can be configured to estimate the pressure of the eye 112 based on the detected air pressure transient.

In a system according to an exemplifying and non-limiting embodiment, the detector 102 comprises means for Schlieren imaging or combined Schlieren and streak imaging to detect a change that takes place in a line integral around a closed curve of the velocity field of the travelling air vortex ring when the travelling air vortex ring contacts the surface of the eye. The closed curve can be e.g. around the theta-axis of the travelling air vortex ring. The theta-axis is perpendicular to the plane of the air vortex ring and parallel with the travelling direction of the air vortex ring. In this exemplifying case, the processing device 103 is configured to estimate the pressure of the eye 112 based on the detected change of the above-mentioned line integral.

It is to be noted that the above-presented technical solutions are non-limiting examples only, and other technical solutions for producing an estimate of the eye pressure based on the interaction between the travelling air vortex ring 111 and the surface of the eye 112 are also possible. Furthermore, in exemplifying and non-limiting embodiments, two or more different technical solutions are used to produce two or more estimates of the eye pressure in order to improve the reliability and the accuracy of the pressure measurement. The final estimate of the eye pressure can be derived with e.g. a predetermined mathematical rule based on two or more estimates obtained with two or more different technical solutions. The final estimate can be e.g. an arithmetic mean of the two or more estimates obtained with the two or more technical solutions.

The processing device 103 can be implemented with one or more processor circuits, each of which can be a programmable processor circuit provided with appropriate software, a dedicated hardware processor such as for example an application specific integrated circuit “ASIC”, or a configurable hardware processor such as for example a field programmable gate array “FPGA”. The software may comprise e.g. firmware that is a specific class of computer software that provides low-level control for hardware of the processing device 103. The firmware can be e.g. open-source software. Furthermore, the processing device 103 may comprise one or more memory circuits each of which can be for example a random-access-memory “RAM” circuit.

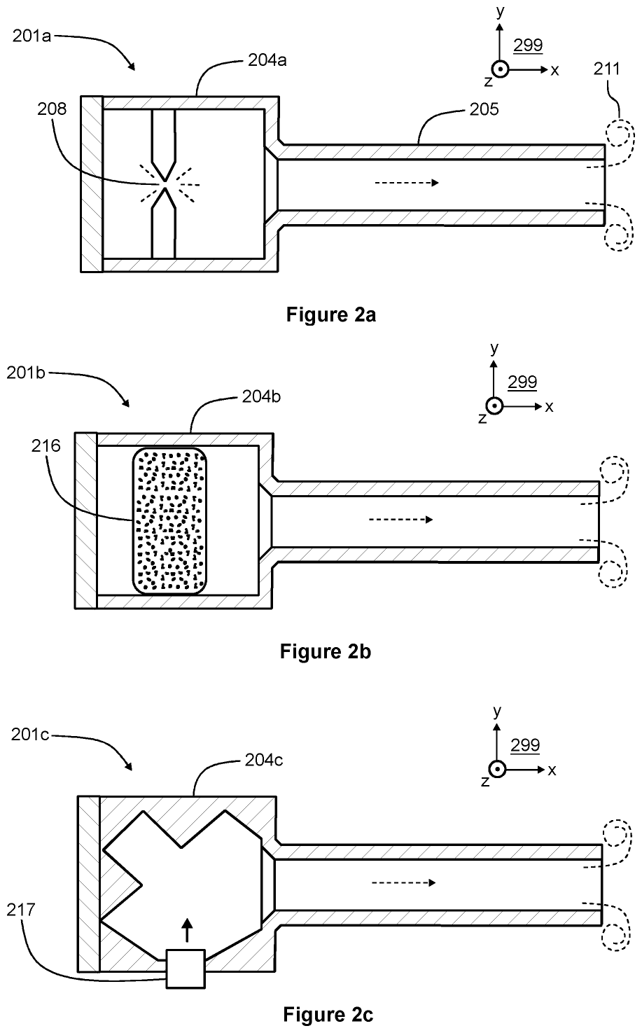

FIG. 2a shows a section view of an excitation source 201 of a system according to an exemplifying and non-limiting embodiment for measuring pressure of an eye. The section plane is parallel with the xy-plane of a coordinate system 299. The excitation source 201a comprises an air pressure pulse source 204a and a flow guide 205 that produces a travelling air vortex ring 211. The formation of the air vortex ring 211 is illustrated with dashed arched lines in FIG. 2a. The air vortex ring 211 is substantially rotationally symmetric with respect to a geometric line parallel with the x-axis of the coordinate system 299. In this exemplifying case, the flow guide 205 comprises a tube having an open end for producing the travelling air vortex ring 211 and the air pressure pulse source 204a comprises a chamber connected to the flow guide 205 and containing a spark gap 208 for producing an air pressure pulse by an electric spark.

FIG. 2b shows a section view of an excitation source 201b of a system according to an exemplifying and non-limiting embodiment for measuring pressure of an eye. In this exemplifying case, an air pressure pulse source 204b comprises a chamber connected to a flow guide and containing chemical substances 216 for producing an air pressure pulse by a chemical reaction between the chemical substances.

FIG. 2c shows a section view of an excitation source 201c of a system according to an exemplifying and non-limiting embodiment for measuring pressure of an eye. In this exemplifying case, an air pressure pulse source 204c comprises a laser source 217 for producing a plasma expansion in a chamber connected to a flow guide to produce an air pressure pulse.

FIG. 2d shows a section view of an excitation source 201d of an exemplifying system for measuring pressure of an eye. In this exemplifying case, an air pressure pulse source 204d comprises a piezo-actuated blower 218 connected to a flow guide and for generating an air pressure pulse.

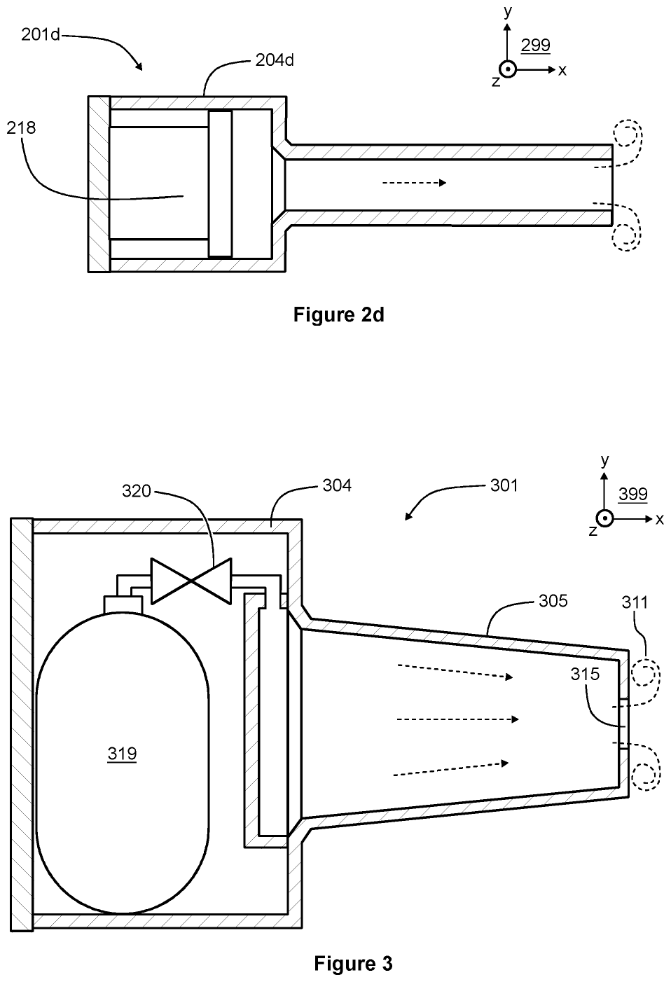

FIG. 3 shows a section view of an excitation source 301 of an exemplifying system for measuring pressure of an eye. The section plane is parallel with the xy-plane of a coordinate system 399. The excitation source 301 comprises an air pressure pulse source 304 and a flow guide 305 for forming a travelling air vortex ring 311. The formation of the air vortex ring 311 is illustrated with dashed arched lines in FIG. 3. The air vortex ring 311 is substantially rotationally symmetric with respect to a geometric line parallel with the x-axis of the coordinate system 399. In this exemplifying case, the flow guide 305 comprises a flow guide chamber having an aperture 315 in a wall of the flow guide chamber. The flow guide chamber has a shape of a truncated cone and an end-wall of the smaller end of the flow guide chamber comprises the aperture 315 and the larger end of the flow guide chamber is connected to the air pressure pulse source 304. In this exemplifying case, the air pressure pulse source 304 comprises a pressure chamber 319 containing pressurized air, e.g. a replaceable pressure air cartridge, and a valve 320 for releasing an air pressure pulse from the pressure chamber 319 to the flow guide 305.

In the above-mentioned examples, the flow guide can be e.g. a mere aperture at a wall of the air pressure pulse source.

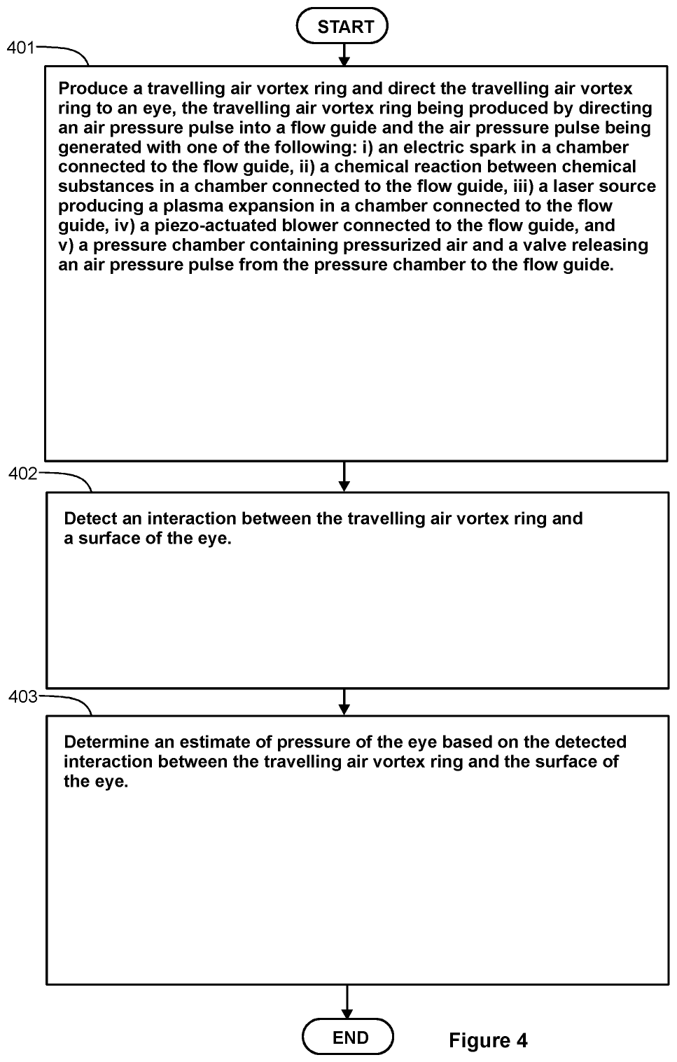

FIG. 4 shows a flowchart of a method according to an exemplifying and non-limiting embodiment for measuring pressure of an eye. The method comprises the following actions:
action 401: producing a travelling air vortex ring and directing the travelling air vortex ring to the eye,
action 402: detecting an interaction between the travelling air vortex ring and a surface of the eye, and
action 403: determining an estimate of the pressure of the eye based on the detected interaction between the travelling air vortex ring and the surface of the eye.

The travelling air vortex ring is produced by directing an air pressure pulse into a flow guide. The air pressure pulse is generated with one of the following: i) an electric spark in a chamber connected to the flow guide, ii) a chemical reaction between chemical substances in a chamber connected to the flow guide, iii) a laser source producing a plasma expansion in a chamber connected to the flow guide, iv) a piezo-actuated blower connected to the flow guide, and v) a pressure chamber containing pressurized air and a valve releasing the air pressure pulse from the pressure chamber to the flow guide.

In a method according to an exemplifying and non-limiting embodiment, the travelling air vortex ring is produced at a place at least 5 cm away from the surface of the eye. In a method according to an exemplifying and non-limiting embodiment, the travelling air vortex ring is produced at a place at least 7.5 cm away from the surface of the eye. In a method according to an exemplifying and non-limiting embodiment, the travelling air vortex ring is produced at a place at least 10 cm away from the surface of the eye.

In a method according to an exemplifying and non-limiting embodiment, the flow guide comprises a tube directed towards the eye. In a method according to another exemplifying and non-limiting embodiment, the flow guide comprises a flow guide chamber having an aperture in a wall of the flow guide chamber so that the aperture is facing towards the eye. In a method according to an exemplifying and non-limiting embodiment, the flow guide chamber has a shape of a truncated cone and the end-wall of the smaller end of the flow guide chamber comprises the aperture and the larger end of the flow guide chamber receives the air pressure pulse.

A method according to an exemplifying and non-limiting embodiment comprises detecting a surface wave caused by the travelling air vortex ring on the surface of the eye. In a typical situation, the surface wave is a manifestation of a membrane wave caused by the travelling air vortex ring on the cornea of the eye. The surface wave can be detected with an optical interferometer, an optical coherence tomography device, a laser Doppler vibrometer, an ultrasonic transducer, or some other suitable device. In a method according to an exemplifying and non-limiting embodiment, the estimate of the pressure of the eye is determined based on the travelling speed of the detected surface wave on the surface of the eye.

A method according to an exemplifying and non-limiting embodiment comprises detecting a displacement of the surface of the eye caused by the travelling air vortex ring. The displacement can be detected with an optical interferometer, an optical coherence tomography device, a laser Doppler vibrometer, an ultrasonic transducer, or some other suitable device. In a method according to an exemplifying and non-limiting embodiment, the estimate of the pressure of the eye is determined based on oscillation rate of the detected displacement. In a method according to an exemplifying and non-limiting embodiment, the estimate of the pressure of the eye is determined based on a speed at which the surface of the eye retracts when being hit by the travelling air vortex ring. In a method according to an exemplifying and non-limiting embodiment, the estimate of the pressure of the eye is determined based on a speed at which the retracted surface of the eye moves back towards its normal position. In a method according to an exemplifying and non-limiting embodiment, the estimate of the pressure of the eye is determined based on a delay after which the retracted surface of the eye moves back towards its normal position. In a method according to an exemplifying and non-limiting embodiment, the estimate of the pressure of the eye is determined based on a retraction depth of the surface of the eye when being hit by the travelling air vortex ring.

A method according to an exemplifying and non-limiting embodiment comprises detecting an air pressure transient reflected off the surface of the eye when the travelling air vortex ring hits the surface of the eye. In a method according to an exemplifying and non-limiting embodiment, the estimate of the pressure of the eye is determined based on the detected air pressure transient.

A method according to an exemplifying and non-limiting embodiment comprises detecting a change that takes place in a line integral around a closed curve of the velocity field of the travelling air vortex ring when the travelling air vortex ring contacts the surface of the eye. The closed curve can be e.g. around the theta-axis of the travelling air vortex ring. The theta-axis is perpendicular to the plane of the air vortex ring and parallel with the travelling direction of the air vortex ring. The detection can be carried out e.g. with Schlieren imaging or with combined Schlieren and streak imaging. In a method according to an exemplifying and non-limiting embodiment, the pressure of the eye is estimated based on the detected change of the above-mentioned line integral.

The non-limiting, specific examples provided in the description given above should not be construed as limiting the scope and/or the applicability of the appended claims. Furthermore, any list or group of examples presented in this document is not exhaustive unless otherwise explicitly stated.

1. A system for measuring pressure of an eye, the system comprising:
an excitation source for producing a travelling air vortex ring and for directing the travelling air vortex ring towards the eye,
a detector for detecting an interaction between the travelling air vortex ring and a surface of the eye, and
a processing device for determining an estimate of the pressure of the eye based on the detected interaction between the travelling air vortex ring and the surface of the eye,
wherein the excitation source comprises an air pressure pulse source and a flow guide for forming the travelling air vortex ring, and the air pressure pulse source comprises one of the following: i) a chamber connected to the flow guide and containing a spark gap for producing an air pressure pulse by an electric spark, ii) a chamber connected to the flow guide and containing chemical substances for producing an air pressure pulse by a chemical reaction between the chemical substances, iii) a laser source for producing a plasma expansion in a chamber connected to the flow guide to produce an air pressure pulse.

2. The system according to claim 1, wherein the flow guide comprises a tube having an open end for forming the travelling air vortex ring.

3. The system according to claim 1, wherein the flow guide comprises a flow guide chamber having an aperture in a wall of the flow guide chamber.

4. The system according to claim 3, wherein the flow guide chamber has a shape of a truncated cone and an end-wall of a smaller end of the flow guide chamber comprises the aperture and a larger end of the flow guide chamber is connected to the pressure pulse source.

5. The system according to claim 1, wherein the detector comprises one of the following for detecting a surface wave launched by the travelling air vortex ring on the surface of the eye: an optical interferometer, an optical coherence tomography device, a laser Doppler vibrometer, an ultrasonic transducer.

6. The system according to claim 5, wherein the processing device is configured to determine the estimate of the pressure of the eye based on travelling speed of the detected surface wave on the surface of the eye.

7. The system according to claim 1, wherein the detector comprises one of the following for detecting a displacement of the surface of the eye caused by the travelling air vortex ring: an optical interferometer, an optical coherence tomography device, a laser Doppler vibrometer, an ultrasonic transducer.

8. The system according to claim 7, wherein the processing device is configured to determine the estimate of the pressure of the eye based on oscillation rate of the detected displacement of the surface of the eye.

9. The system according to claim 1, wherein the detector comprises a pressure sensor for detecting an air pressure transient reflected off the surface of the eye when the travelling air vortex ring hits the surface of the eye.

10. The system according to claim 9, wherein the processing device is configured to determine the estimate of the pressure of the eye based on the detected air pressure transient.

11. A method for measuring pressure of an eye, the method comprising:
producing a travelling air vortex ring and directing the travelling air vortex ring to the eye,
detecting an interaction between the travelling air vortex ring and a surface of the eye, and
determining an estimate of the pressure of the eye based on the detected interaction between the travelling air vortex ring and the surface of the eye,
wherein the travelling air vortex ring is produced by directing an air pressure pulse into a flow guide, and the air pressure pulse is generated with one of the following: i) an electric spark in a chamber connected to the flow guide, ii) a chemical reaction between chemical substances in a chamber connected to the flow guide, iii) a laser source producing a plasma expansion in a chamber connected to the flow guide.

12. The system according to claim 2, wherein the detector comprises one of the following for detecting a surface wave launched by the travelling air vortex ring on the surface of the eye: an optical interferometer, an optical coherence tomography device, a laser Doppler vibrometer, an ultrasonic transducer.

13. The system according to claim 3, wherein the detector comprises one of the following for detecting a surface wave launched by the travelling air vortex ring on the surface of the eye: an optical interferometer, an optical coherence tomography device, a laser Doppler vibrometer, an ultrasonic transducer.

14. The system according to claim 4, wherein the detector comprises one of the following for detecting a surface wave launched by the travelling air vortex ring on the surface of the eye: an optical interferometer, an optical coherence tomography device, a laser Doppler vibrometer, an ultrasonic transducer.

15. The system according to claim 2, wherein the detector comprises one of the following for detecting a displacement of the surface of the eye caused by the travelling air vortex ring: an optical interferometer, an optical coherence tomography device, a laser Doppler vibrometer, an ultrasonic transducer.

16. The system according to claim 3, wherein the detector comprises one of the following for detecting a displacement of the surface of the eye caused by the travelling air vortex ring: an optical interferometer, an optical coherence tomography device, a laser Doppler vibrometer, an ultrasonic transducer.

17. The system according to claim 4, wherein the detector comprises one of the following for detecting a displacement of the surface of the eye caused by the travelling air vortex ring: an optical interferometer, an optical coherence tomography device, a laser Doppler vibrometer, an ultrasonic transducer.

18. The system according to claim 2, wherein the detector comprises a pressure sensor for detecting an air pressure transient reflected off the surface of the eye when the travelling air vortex ring hits the surface of the eye.

19. The system according to claim 3, wherein the detector comprises a pressure sensor for detecting an air pressure transient reflected off the surface of the eye when the travelling air vortex ring hits the surface of the eye.

20. The system according to claim 4, wherein the detector comprises a pressure sensor for detecting an air pressure transient reflected off the surface of the eye when the travelling air vortex ring hits the surface of the eye.
